(19) United States
(12) Patent Application Publication
(10) Pub. No.: US 2022/0106582 A1
(43) Pub. Date: Apr. 7, 2022

(54) EXOSOMAL NUCLEIC ACID EXTRACTION METHOD

(71) Applicant: National Central University, Taoyuan (TW)

(72) Inventors: Wen-Yih Chen, Taoyuan City (TW); Hsin-Yi Lin, Taipei City (TW); Chi-Hung Lai, Chiayi City (TW)

(73) Assignee: National Central University, Taoyuan (TW)

(21) Appl. No.: 17/082,026

(22) Filed: Oct. 28, 2020

(30) Foreign Application Priority Data
Oct. 7, 2020 (TW) 109134731

(51) Int. Cl. C12N 15/10

(52) U.S. Cl. CPC C12N 15/1006

(57) ABSTRACT

An exosomal nucleic acid extraction method is provided. A substrate is provided, on which antibodies and silicon nanoparticles are disposed, and the antibodies have binding specificity to exosomal surface antigens. After that, based on the binding specificity between the antibodies and the exosomal surface antigens, exosomes in a specimen are separated. Next, the nucleic acids in the separated exosomes are adsorbed by the silicon nanoparticles.

### CROSS-REFERENCE TO RELATED APPLICATION

This application claims the priority benefit of Taiwan application serial no. 109134731, filed on Oct. 7, 2020. The entirety of the above-mentioned patent application is hereby incorporated by reference herein and made a part of this specification.

### BACKGROUND

### Technical Field

The invention relates to a nucleic acid extraction method, and particularly relates to an exosomal nucleic acid extraction method.

### Description of Related Art

In today's society, early diagnosis and treatment have been generally regarded as better medical practices. Although many advanced countries have developed methods that may accurately detect diseases, there are still obstacles on promotion to be overcome. One is that the detection cannot be carried out quickly and immediately, or in a way of so call point of care testing or point of need testing, and the other is that the equipment required for detection is expensive, which makes it more difficult for the detection to be implemented in developing countries or underdeveloped areas.

In recent years, related studies have pointed out that there is a correlation between human genetic materials and diseases. Exosomes are small vesicles vomited by cells, and there is a high correlation between abnormal nucleic acid expression in the exosomes and some human diseases. In conventional technologies, an ultracentrifugation-based isolation technique or a precipitation method are mostly used for separating the exosomes. These two methods not only require expensive centrifugal equipment, but also the precipitation method has a problem of poor purity in the separated exosomes. Both of the methods have also to be performed in a centralized lab facilities. In addition, the conventional nucleic acid separation method is liquid phase and solid phase separation, but since it is easy to cause product contamination or poor extraction efficiency, a combination of solid and liquid phases is often used for nucleic acid extraction.

Based on the above description, to develop a simple, convenient and rapid exosomal nucleic acid extraction method, which may effectively reduce costs in preparation and purification, is an important subject for current researches.

### SUMMARY

The invention is directed to an exosomal nucleic acid extraction method, which is adapted to easily, conveniently and quickly achieve purposes of separating exosomes and extracting exosomal nucleic acid, and meanwhile reduce separation and purification costs.

The invention provides an exosomal nucleic acid extraction method including following steps. A substrate is provided, and antibodies and silicon nanoparticles are disposed on the substrate, wherein the antibodies have binding specificity to exosomal surface antigens. Thereafter, based on the binding specificity between the antibodies and the exosomal surface antigens, the exosomes in a specimen (tissue samples) are separated. Then, nucleic acids in the separated exosomes are adsorbed by the silicon nanoparticles on the substrate.

In an embodiment of the invention, the substrate includes a paper substrate or a porous fiber substrate.

In an embodiment of the invention, the antibodies are immobilized on the substrate by chemical bonding or physical adsorption.

In an embodiment of the invention, the silicon nanoparticles are disposed on the substrate by aqueous solution coating, spraying, adsorption or a sol-gel method.

In an embodiment of the invention, the antibodies and the silicon nanoparticles are disposed in different regions on the substrate.

In an embodiment of the invention, there is a flow channel connected between the different regions configured with the antibodies and the silicon nanoparticles.

In an embodiment of the invention, the specimen flows from the region where the antibodies are disposed to the region where the silicon nanoparticles are disposed through the flow channel.

In an embodiment of the invention, after the exosomes in the specimen are separated by the antibodies, the exosomes are lysed by a lysis solution. After the exosomes are lysed, the nucleic acids in the separated exosomes are adsorbed by the silicon nanoparticles.

In an embodiment of the invention, after the nucleic acids in the separated exosomes are adsorbed by the silicon nanoparticles, a region adsorbed with the nucleic acids on the substrate is cut off and placed in eluent to complete nucleic acid extraction of the exosomes.

Based on the above description, the invention provides an exosomal nucleic acid extraction method, in which by integrating affinity between the antibodies and the exosomal surface antigens and the adsorption mechanism of the nucleic acids and the silicon nanoparticles on the same substrate, the method may easily, conveniently and quickly achieve purposes of separating the exosomes and extracting the exosomal nucleic acids, and meanwhile reduce the separation and purification costs. Therefore, the disadvantages of expensive separation equipment and professionals required for exosomal nucleic acid extraction in the prior art are mitigated, and the cumbersome sample processing in the prior art is avoided.

### DESCRIPTION OF THE EMBODIMENTS

Hereinafter, embodiments of the invention will be described in detail. However, these embodiments are illustrative, and the invention is not limited thereto. In the following, the terms used in the specification are defined and explained first.

“Specimen” refers to a sample to be tested. For example, the specimen may be a sample extracted from body fluids such as blood, urine, saliva, or from a cell culture fluid.

“Nucleic acid” is a biological molecule that exists in a cell nucleus, which is one of the most basic substances that constitutes a living body, and is responsible for preservation and transmission of genetic information in the organism, and exists in all animals, plants, viruses, and phages. Nucleic acids are mainly divided into two categories, which are respectively deoxyribonucleic acids (DNA) and ribonucleic acids (RNA) according to their chemical structures.

“Exosome” is a nanovesicle composed of a lipid bilayer, which comes from a multivesicular body of invagination of a cell membrane, this type of vesicle is formed in the cell, and different from a general budding method, a formation process thereof is that a mother cell loads biomolecules into intraluminal vesicles (ILVs) formed through invagination of the cell membrane, so as to form multivesicular bodies (MVBs) containing the intraluminal vesicles. Thereafter, the multivesicular bodies are merged with the mother cell membrane at a specific location and are released outside the cell, and are widely distributed in body fluids such as blood, saliva, breast milk, and urine. Due to the special functions of the exosome, it has potential application values, on the one hand, it may be used as a diagnostic marker for various diseases, and on the other hand, it may be used as a means of treatment.

The invention provides an exosomal nucleic acid extraction method, which basically includes following steps. A substrate is provided, and antibodies and silicon nanoparticles are disposed on the substrate, where the antibodies have binding specificity to exosomal surface antigens. Thereafter, based on the binding specificity between the antibodies and the exosomal surface antigens, the exosomes in a specimen are separated. Then, nucleic acids in the separated exosomes are adsorbed by the silicon nanoparticles. Hereinafter, the above steps are described in detail.

### <Provide the Substrate>

The substrate of the invention may include, but is not limited to, a paper substrate or a porous fiber substrate. The paper substrate is preferably Whatman grade 1 filter paper, but the invention is not limited thereto. Since the invention uses the paper substrate or the porous fiber substrate, it may reduce the manufacturing cost, and through a capillarity phenomenon, a fluid flowing effect may be achieved without intervention of any external force, which may effectively mitigate the disadvantages of expensive separation equipment and professionals required for exosomal nucleic acid extraction in the past, and avoid the cumbersome sample processing in the past.

### <Dispose Antibodies on the Substrate>

In the embodiment, the antibodies are immobilized on the substrate by chemical bonding or physical adsorption. The antibodies have binding specificity to the exosomal surface antigens. Therefore, based on the binding specificity between the antibodies and the exosomal surface antigens, the exosomes in the specimen are separated.

In detail, in the invention, the antibodies may be, for example, immobilized on the substrate through the following methods, but the invention is not limited thereto, and other chemical bonding or physical adsorption methods may also be used to immobilize the antibodies on the substrate. First, the substrate is immersed in a 3-mercaptopropyl trimethoxysilane solution, and left to stand at a room temperature to proceed a silanization reaction, and then it is hydrogen-bonded with hydroxyl groups on a substrate surface and dehydrated to form a C—O—Si bond. Thereafter, the substrate is immersed in an N-γ-maleimidobutyryloxy succinimide ester (GMBS) solution, and left to stand at room temperature to let GMBS and —SH to proceed an addition reaction to form a bond. Then, the substrate is immersed in a NeutrAvidin solution and left to stand at a temperature of 4° C. to let neutravidin and the GMBS to form an amide bond. Thereafter, the substrate is immersed in a BSA blocking solution to let BSA to cover functional groups that have not been transplanted successfully to avoid non-specific adsorption. Finally, the substrate is immersed in a solution containing the antibodies, and the antibodies are immobilized on the substrate through strong affinity between the neutravidin and biotin.

### <Dispose Silicon Nanoparticles on the Substrate>

In the embodiment, the silicon nanoparticles are disposed on the substrate by aqueous solution coating, spraying, adsorption or the sol-gel method. In detail, the substrate is first cut into an appropriate size, and about 20 μL of silicon nanoparticle solution (about 3 mg/mL in dH2O) is respectively taken from upper and lower sides of an experimental region to perform the step of coating the silicon nanoparticles, and drying the same at room temperature. The sol gel method was used for formation of a 3D network through hydrolysis and condensation reactions. 0.1 g silicon nanoparticles as the precursor were added into ddH2O and stirred at 60° C. for 10 mins. 0.05 g silicon nanoparticles were then added in sol gel solution after being mixed with 30 μL HCl solution as acid catalyst and stirred at 80° C. for 30 mins. 5 μL sol gel solution was finally taken from upper and lower sides of a cellulose paper following with drying by evaporation at 70° C. for 60 mins for shrinkage and collapse.

### <An Operation Process of Exosomal Nucleic Acid Extraction>

The operation process of the exosomal nucleic acid extraction of the invention may be carried out through different device configurations. The operation process of the exosomal nucleic acid extraction of the invention is described below in different embodiments.

In a first embodiment, the antibodies and the silicon nanoparticles are disposed in different regions on the substrate. These two different regions may produce a hydrophobic region and a hydrophilic region through a waterproof tape to achieve various flow channel designs. Alternatively, liquid flow may be controlled through a paper bridge, and the paper bridge is composed of a piece of acrylic board bonded with paper through a double-sided tape. The liquid may flow between the two regions by switching on the paper bridge, and if the paper bridge is not switched on (switched off), the liquid cannot flow between the two regions. There is a flow channel connected between the different regions configured with the antibodies and the silicon nanoparticles, and the specimen flows from the region configured with the antibodies to the region configured with the silicon nanoparticles through the flow channel. However, the invention is not limited thereto, and the operation process of exosomal nucleic acid extraction may also be performed without using the design of the flow channel (which is a mechanism adopted in a second embodiment, and is described in detail below).

To be specific, the specimen is first added to an injection port on the substrate, and flows to the region configured with the antibodies on the substrate along the flow channel. At this time, based on the binding specificity between the antibodies and the exosomal surface antigens on the substrate, the exosomes in the specimen are separated, and the other biomolecules are removed through an absorbent pad. After the exosomes in the specimen are separated through the antibodies, a lysis solution is added from the injection port, and the exosomes are lysed by the lysis solution. After the exosomes are lysed, the exosomal lysis solution flows from the region configured with the antibodies to the region configured with the silicon nanoparticles on the substrate along the flow channel. Thereafter, the nucleic acids in the separated exosomes are adsorbed by the silicon nanoparticles on the substrate, and the other biomolecules are removed through the absorbent pad. Then, the region adsorbed with the nucleic acids on the substrate is cut off and placed in eluent to complete the nucleic acid extraction of the exosomes.

An operating mechanism of the second embodiment is basically similar to that of the above-mentioned first embodiment, and the difference is that there is no flow channel design, i.e., there is no flow channel connected between the different regions configured with the antibodies and the silicon nanoparticles. To be specific, the specimen is first added to the region configured with the antibodies on the substrate, and the exosomes in the specimen are separated based on the binding specificity between the antibodies and the exosomal surface antigens on the substrate. Then, a rinsing solution is added above the region configured with the antibodies on the substrate to rinse off substances that are not bound to the antibodies. Thereafter, the region configured with the antibodies on the substrate is placed into the lysis solution to lyse the exosomes with the lysis solution. The obtained exosomal lysis solution is taken out through a dropper, and dropped into the region configured with the silicon nanoparticles. Then, the nucleic acids in the separated exosomes are adsorbed by the silicon nanoparticles on the substrate, and the region adsorbed with the nucleic acids on the substrate is cut off and placed in the eluent to complete the exosomal nucleic acid extraction.

Since molecular forces between the antibodies and the antigens include hydrogen bonds, Van der Waals forces, hydrophobic forces, and ionic bonds, an affinity constant between the antibodies and the antigens is easily affected by temperature, pH value and solvent, so that the affinities between the antibodies and antigens are different. The invention chooses to perform a step of bonding the antibodies and the antigens at room temperature, which is more helpful to an application of point-of-care diagnostics (POCT), and uses PBS to neutralize the pH value of the specimen, so as to avoid affecting bonding of the antibodies and the antigens due to the different pH values of the specimen.

In the aqueous solution, silanol groups may be formed on surfaces of the silicon nanoparticles, and pka of silicon dioxide is about 5 to 8 according to the type of the silanol groups on the surfaces, so that the pH value of the silanol groups is between 5 and 8, as the pH value of the silanol groups increases, a degree of hydroxyl deprotonation on the surfaces increases, and surface negative charges are also increased.

In order to maximize the nucleic acid adsorption and desorption of the silicon nanoparticles on the substrate, in case of the nucleic acid adsorption, an aqueous solution of pH4 is used as a binding buffer, and the nucleic acid adsorption reaction of the silicon nanoparticles on the substrate is carried out at room temperature. Protonated surfaces of the silanol groups have many hydroxyl groups, which reduces a negative repulsive force between the surfaces and the nucleic acids, and provides more hydrogen bonding positions, so that an amount of nucleic acid adsorption is increased, and since the adsorption effect between the nucleic acids and the silicon nanoparticles under the pH4 environment is an ionic effect based on electrostatic force, under the general room temperature, a bonding ability there between is not much affected by temperature changes. The invention chooses to directly carry out the nucleic acid adsorption at room temperature, which is more helpful to the application of point-of-care diagnostics/testing (POCT). In the embodiment, ddH2O may also be used as the binding buffer.

In case of the nucleic acid desorption, a pH9 aqueous solution may be used as an elution buffer, and the nucleic acid desorption is carried out at the temperature 55° C. for 45 minutes. As a degree of deprotonation of the silanol groups on the surfaces increases, the negative repulsive force increases, and heating breaks the hydrogen bond between the nucleic acids and the silicon nanoparticles, which increases the amount of nucleic acid desorption, and the amount of desorption at the temperature of 55° C. is higher than that at the room temperature. In the embodiment, the ddH2O may also be used as the elution buffer.

In summary, the invention provides the exosomal nucleic acid extraction method, in which by integrating affinity between the antibodies and the exosomal surface antigens and the adsorption mechanism of the nucleic acids and the silicon nanoparticles on the same substrate, the method may easily, conveniently and quickly achieve purposes of separating the exosomes and extracting the exosomal nucleic acids, and meanwhile reduce the manufacturing costs. Therefore, the disadvantages of expensive separation equipment and professionals required for exosomal nucleic acid extraction in the past are mitigated, and the cumbersome sample processing in the past is avoided, and also the idea of applying POCT in daily life is implemented. In addition, since the exosomes in the specimen are separated by using immunoassay, it is more specific compared with the conventional centrifugal method or precipitation method, and in collaboration with regulation of the absorption and desorption mechanism between the nucleic acids and the silicon nanoparticles, the maximum nucleic acid adsorption and desorption is achieved. Since the paper substrate or the porous fiber substrate may be used, the effect of fluid flow may be achieved through the capillary phenomenon, which saves a need for a fluid pressurization device.

What is claimed is:

1. An exosomal nucleic acid extraction method, comprising:
providing a substrate, and disposing antibodies and silicon nanoparticles on the substrate, wherein the antibodies have binding specificity to exosomal surface antigens;
separating exosomes in a specimen based on the binding specificity between the antibodies and the exosomal surface antigens; and
adsorbing nucleic acids in the separated exosomes by the silicon nanoparticles.

2. The exosomal nucleic acid extraction method as claimed in claim 1, wherein the substrate comprises a paper substrate or a porous fiber substrate.

3. The exosomal nucleic acid extraction method as claimed in claim 1, wherein the antibodies are immobilized on the substrate by chemical bonding or physical adsorption.

4. The exosomal nucleic acid extraction method as claimed in claim 1, wherein the silicon nanoparticles are disposed on the substrate by aqueous solution coating, spraying or a sol-gel method.

5. The exosomal nucleic acid extraction method as claimed in claim 1, wherein the antibodies and the silicon nanoparticles are disposed in different regions on the substrate.

6. The exosomal nucleic acid extraction method as claimed in claim 1, wherein after the exosomes in the specimen are separated by the antibodies, the exosomes are lysed by a lysis solution, and after the exosomes are lysed, the nucleic acids in the separated exosomes are adsorbed by the silicon nanoparticles.

7. The exosomal nucleic acid extraction method as claimed in claim 1, wherein after the nucleic acids in the separated exosomes are adsorbed by the silicon nanoparticles, a region adsorbed with the nucleic acids on the substrate is cut off and placed in eluent to complete nucleic acid extraction of the exosomes.
